(19) United States
(12) Patent Application Publication
(10) Pub. No.: US 2002/0018778 A1
(43) Pub. Date: Feb. 14, 2002

(54) Passive desensitization

(76) Inventor: Michael J. Caplan, Woodbridge, CT (US)

Correspondence Address: C. Hunter Baker, M.D., Ph.D., Choate, Hall & Stewart, 53 State Street, Exchange Place, Boston, MA, 02109, US

(21) Appl. No.: 09/731,221

(22) Filed: Dec. 6, 2000

Related U.S. Application Data
(63) Continuation-in-part of application No. 09/455,294, filed on Dec. 6, 1999.
(60) Provisional application No. 60/213,765, filed on Jun. 23, 2000.
(60) Provisional application No. 60/235,797, filed on Sep. 27, 2000.

(51) Int. Cl. A61K 39/00; A61K 38/19

(52) U.S. Cl. 424/184.1; 424/85.1

(57) ABSTRACT

IgE-blocking agents and methods of their use have been developed for desensitizing an individual to an antigen. These IgE-blocking agents work by blocking the antigen-binding site of the IgE molecules and thereby preventing the antigen from binding. These agents typically have up to one IgE binding site present per molecule so as prevent any cross-linking of IgE which could lead to an allergic reaction. Methods of using these novel IgE blocking agents include administering the agents to alleviate or prevent allergic reactions as well as administering the agents to decrease the risk of allergic reactions during immunotherapy or “rush” immunotherapy. Compositions and kits comprising these IgE binding agents are also provided.

### RELATED APPLICATIONS

The present application is a continuation-in-part of co-pending application U.S. Ser. No. 09/455,294, filed Dec. 6, 1999, which is incorporated herein by reference in its entirety. The present application also claims priority to co-pending provisional applications, U.S. Ser. No. 60/213,765, filed Jun. 23, 2000, and U.S. Ser. No. 60/235,797, filed Sep. 27, 2000, each of which is incorporated herein by reference.

### BACKGROUND OF THE INVENTION

Allergic and automatic reactions pose serious public health problems worldwide. Pollen allergy alone (allergic rhinitis or hay fever) affects about 10-15% of the population, and generates huge economic costs. For example, reports estimate that pollen allergy generated $1.8 billion of direct and indirect expenses in the United States in 1990 (Fact Sheet, National Institute of Allergy and Infectious Diseases, www.miaid.nih. gov/factsheets/allergystat.html; McMenamin, Annals of Allergy 73:35, 1994; each of which is incorporated herein by reference). More serious than the economic costs associated with pollen and other inhaled allergens (e.g., molds, dust mites, animal danders) is the risk of anaphylactic reaction observed with allergens such as food allergens, insect venoms, drugs, and latex.

Allergic reactions occur when an individual's immune system overreacts, or reacts inappropriately, to an encountered antigen. No allergic reaction is believed to occur the first time an individual is exposed to a particular antigen. However, the initial immune response to an antigen primes the system for subsequent allergic reactions. In particular, the antigen is taken up by antigen presenting cells (e.g., macrophages or dendritic cells) that degrade the antigen and then display antigen fragments to T cells. The activated T cells respond by secreting a collection of cytokines that have effects on other cells of the immune system. The profile of cytokines secreted by responding T cells determines whether subsequent exposures to the particular antigen will induce allergic reactions. When T cells respond by secreting interleukin-4 (IL-4), the effect is to stimulate the maturation of B cells that produce IgE antibodies specific for the antigen. These antigen-specific IgE antibodies then attach to specific receptors on the surface of mast cells and basophils, where they act as a trigger to initiate a rapid reaction to the next exposure to the antigen.

When the individual next encounters the antigen, it is quickly bound by these surface-associated IgE molecules. Each antigen typically has more than one IgE binding site, so that the surface-bound IgE molecules quickly become crosslinked to one another through their simultaneous (direct or indirect) associations with antigen. Such cross-linking induces mast cell degranulation, resulting in the release of histamines and other substances that induce the symptoms associated with allergic reaction. Individuals with high levels of IgE antibodies are known to be particularly prone to allergies.

One approach to treating allergies is antigen immunotherapy which attempts to “vaccinate” a sensitive individual against a particular allergen by periodically injecting or treating the individual with a crude suspension of the raw allergen. The goal is to modulate the allergic response mounted in the individual through controlled administration of known amounts of antigen. If the therapy is successful, the individual's allergic response is diminished, or can even disappear. However, the therapy can require several rounds of vaccination, over an extended time period (3-5 years), and very often does not produce the desired results. Moreover, certain individuals suffer anaphylactic reactions to the vaccines, despite their intentional, controlled administration.

Another more commonly used approach to treating allergies is the administration of histamine antagonists. These drugs are widely available in over-the-counter formulation, but unfortunately they merely ameliorate the symptoms of the allergic response rather than providing for any type of permanent cure or protection against recurrence.

There is a need for the development of improved treatments and prevention of allergic reactions, particularly anaphylactic reactions. Also, improved methods of desensitizing individuals allergic to particular antigens are needed.

### SUMMARY OF THE INVENTION

The present invention provides a system for desensitizing an individual known to be sensitive to an antigen. According to the invention, an agent that specifically blocks the antigen-binding sites on the offending anti-antigen IgE molecules is administered to the individual. The agent is said to “passively desensitize” the individual to the antigen because it is thought to act via a biochemical competition rather than via an “active” immunomodulation. In some embodiments of the invention, passive desensitization blocking inventive agents may additionally have active effects on the immune system (e.g., may induce a shift from a Th2-type response to a Th1-type responses) to a give antigen. However, such active immunomodulation is not required.

Preferred invention blocking agents are characterized in that their administration does not result in cross-linking of anti-antigen IgE, and in that, after exposure to the agent, the individual's antigen sensitivity is at least temporarily reduced. In particular, competition between the blocking agent and the antigen is expected to reduce the amount of antigen-IgE complexation, and therefore to reduce the extent of cross-linking of anti-antigen IgE molecules. Such reduced cross linking should result in reduced symptoms of sensitivity (e.g., reduced histamine release, etc.). Administration of inventive blocking agents may be accomplished using any means known in the art; preferred modes of administration include, but are not limited to, intravenous, oral, transdermal, intradermal, or intranasal administration. Preferably, an excess of blocking agent is administered, so that a significant reduction in antigen binding is observed. One advantage of the present invention is the resulting reduced risk of allergic reactions, including anaphylaxis, when the antigen to which the IgE are directed is administered after the IgEs have been blocked.

In certain preferred embodiments of the invention, the agent to be used to specifically block the IgE comprises one or more antigen fragments. In particularly preferred embodiments, the agent comprises a collection of fragments (e.g., peptides), each of which has one and only one functional IgE binding site so that it can block the binding site of an IgE molecule but cannot cross-link IgE molecules and thereby lead to activation of a mast cell. A collection of antigen fragments that contain only one IgE binding site has the advantage of blocking only those IgE molecules that bind the offending antigen and hence contribute to the risk of an allergic response. Other preferred IgE-blocking agents include immunoglobulins and fragments thereof (e.g., monovalent immunoglobulin), as well as small molecules.

In some preferred embodiments, the inventive blocking agent is sufficient to modulate the immune response and provides active as well as passive desensitization. Alternatively or additionally, the agent may be administered with adjuvants, cytokines, and/or inducing agents to induce active desensitization. For example, the agent may be administered together with antigen fragments (e.g., peptides if the antigen is a protein antigen and therefore may have modulatory effects on the immune response that contain no IgE binding sites but do contain T-cell epitopes.

Preferably, administration of an inventive passive desensitization composition (i.e., a composition comprising on effective amounts of a blocking agent) can protect an individual against, subsequent inadventent or intentional exposures to antigen. Thus, prior to exposure, or risk of exposure, to the allergic antigen may receive (e.g., by self-administration or through administration by a friend, relative, a acquaintance, or a health care professional, an inventive blocking agent. For example, if a patient is allergic to peanuts, he may administer a blocking agent to himself before eating at a Chinese restaurant that may serve foods prepared using peanut oil. In another example, a patient who is allergic to pollen may administer to herself the IgE-blocking agents repeatedly over weeks to months until the pollen season is over and thereby prevent a flare up of allergies. In yet another example, an individual may receive administration of a blocking agent before a potential exposure to the antigen (e.g., eating a chocolate bar which may inadvertently contain peanut components).

In another embodiment of the invention, an individual allergic to a needed drug may be treated with a blocking agent to prevent an allergic response upon administration of the drug. For example, if a patient was allergic to penicillin but needed a penicillin-type antibiotic to treat an infection, the appropriate blocking agent could be administered to prevent or lessen an allergic response upon administration of the needed antibiotic.

In another aspect of the present invention, administration of the inventive agent is followed by subsequent exposure to the antigen, or portions thereof, in the hope of eliciting tolerance to the antigen. In certain preferred embodiments, antigen exposure takes the form of standard immunotherapy or rush immunotherapy. Rush immunotherapy is typically performed to achieve tolerance to an antigen and is done with more antigen than is typically used in standard immunotherapy. Preferably, exposure to the antigen occurs within the passive desensitization period. For example, in many preferred embodiments, antigen exposure occurs within 48 hours, more preferably within 24 hours, and most preferably within 4-8 hours or less. Exposure to antigen may also involve exposure to cytokines, adjuvants, or inducing agents. Where exposure is to standard or rush immunotherapy, it preferably occurs in a hospital setting or other suitably equipped medical facility.

The present invention also provides for compositions and kits to be used in the passive desensitization method and passive desensitization/immunotherapy method.

### Definitions

“Allergen”: An “allergen” is an antigen that (i) elicits an IgE response in an individual; and/or (ii) elicits an asthmatic reaction (e.g., chronic airway inflammation characterized by eosinophilia, airway hyperresponsiveness, and excess mucus production), whether or not such a reaction includes a detectable IgE response. Preferred allergens for the purpose of the present invention are protein allergens, although the invention is not limited to such. An exemplary list of protein allergens is presented as an Appendix in U.S. patent application (U.S. Ser. No. 09/455,294), filed Dec. 6, 1999, which is incorporated herein by reference. This list was adapted on Jul. 22, 1999, from ftp://biobase.dk/pub/who-iuis/allergen.list, which provides lists of known allergens.

“Allergic reaction”: An allergic reaction is a clinical response by an individual to an antigen. Symptoms of allergic reactions can affect the cutaneous (e.g., urticaria, angioedema, pruritus), respiratory (e.g., wheezing, coughing, laryngeal edema, rhinorrhea, watery/itching eyes), gastrointestinal (e.g., vomiting, abdominal pain, diarrhea), and/or cardiovascular (if a systemic reaction occurs) systems. For the purposes of the present invention, an asthmatic reaction is considered to be a form of allergic reaction.

“Anaphylactic antigen”: An “anaphylactic antigen” according to the present invention is an antigen that is recognized to present a risk of anaphylactic reaction in allergic individuals when encountered in its natural state, under natural conditions. For example, for the purposes of the present invention, pollens and animal danders or excretions (e.g., saliva, feces, urine) are not considered to be anaphylactic antigens. On the other hand, some food antigens, insect antigens, drugs, and rubber (e.g., latex) antigens are generally considered to be anaphylactic antigens. Food antigens are particularly preferred anaphylactic antigens for use in the practice of the present invention. Particularly interesting anaphylactic antigens are those (e.g., peanuts, tree nuts, seeds, insect venom, seafood, shellfish, and fish) to which reactions are commonly so severe as to create a risk of death.

“Anaphylaxis” or “anaphylactic reaction”: “Anaphylaxis” or “anaphylactic reaction”, as used herein, refers to an immune response characterized by mast cell degranulation secondary to antigen-induced cross-linking of the high-affinity IgE receptor on mast cells with subsequent mediator release and the production of pathological responses in target organs, e.g., airway, skin, digestive tract, and cardiovascular system. As is known in the art, the severity of an anaphylactic reaction may be monitored, for example, by assaying cutaneous reactions, puffiness around the eyes and mouth, and/or diarrhea, followed by respiratory reactions such as wheezing and labored respiration. The most severe anaphylactic reactions can result in loss of consciousness and/or death.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Antigen”: An “antigen” is (i) any compound or composition that elicits an immune response; and/or (ii) any compound that binds to a T cell receptor (e.g., when presented by an MHC molecule) or to an antibody produced by a B-cell. Those of ordinary skill in the art will appreciate that an antigen may be a collection of different chemical compounds (e.g., a crude extract or preparation) or a single compound (e.g., a protein). Preferred antigens are protein antigens, but antigens need not be proteins for the practice of the present invention. Other preferred antigens are small molecules such as drugs (e.g., penicillin).

“Antigen presenting cell”: An APC is any cell that is capable of presenting antigen in a manner sufficient to induce an immune response in a naive cell or to stimulate an immune response in a previously primed cell. A “professional” APC (pAPC) is an APC that displays antigen in the context of an MHC molecule and (i) is capable of providing co-stimulatory signals and initiating a primary immune response (i.e., activating or priming a naive T cell); and/or (ii) expresses cytokines sufficient to induce an immune response in a committed T cell. Such pAPCs include macrophages, dendritic cells, and B cells.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Block”: The term “block” when referring to blocking of an IgE refers to an agent's ability to compete with antigen for binding to an anti-antigen IgE molecule. This ability may be assayed by any available means such as, for example, direct binding studies or indirect competition studies. For example, an agent is considered to block the IgE binding site if (i) its affinity for anti-antigen IgE is at least about 2-5 fold, preferably at least about 10, 20, 50, or 100 fold higher than that observed for natural antigen; (ii) its kinetics of the interaction with the anti-antigen IgE is such that the on rate is 2-5 fold, preferably at least about 10, 50, or 100-fold faster than that observed for natural antigen and/or the off rate is 2-5 fold, preferably at least about 10, 50, or 100-fold slower than that observed for natural antigen; (iii) the level of IgE binding to natural antigen is reduced at least about 10%, preferably at least about 20, 50, 80, 90, or 95%, in the presence of the agent; (iv) its concentration is high enough to preferentially bind the IgE binding site over the natural antigen; (v) mast cells containing surface-bound anti-antigen IgE degranulate less when exposed to antigen (at least about 2 fold, preferably at least about 3, 5, 10, 20, 50, or 100 fold less) in the presence of agent as compared with in its absence; and/or (vi) individuals contacted with agent develop fewer (at least about 2 fold, preferably at least about 3, 5, 10, 20, 50, or 100 fold fewer) allergic symptoms, or develop symptoms that are reduced in severity or intensity, when they have been exposed to blocking agent prior to exposure to antigen.

“Cytokine”: A “cytokine” is a small molecule that is released from or expressed by a cell and can alter the behavior or regulate the activity of one or more immunologically relevant target cells expressing a receptor for the cytokine. Cytokines that, if expressed by a pAPC or other cell during presentation of antigen to a T cell, would induce a particular response in that T cell can be classified according to the type of response they induce in the T cell. For example, cytokines that induce a Th1 response (e.g., IL-12, IL-2, IL-18, IL-1 or fragments thereof, IFN, and/or IFN, etc.) are referred to herein as “Th1 stimulating cytokines”; cytokines such that induce a Th2 response (e.g., IL-4, etc.) are referred to herein as “Th2 stimulating cytokines”. Cytokines that are produced during a Th1 response (e.g., IFN, TNF, etc.) are referred to as “Th1 cytokines”; cytokines that are produced during a Th2 response (e.g., IL-4, IL-5, etc.) are referred to as “Th2 cytokines”.

“Effective amount”: In general, the “effective amount” of an active ingredient (e.g., a blocking agent, a blocking agent component, an antigen, etc.) refers to the amount necessary to elicit the desired biological response. For example, the effective amount of blocking agent in a desensitizing composition is the amount that, when administered to an individual who is sensitized to a particular antigen prior to exposure to the antigen, results in a reduction in symptoms (nature, extent, and/or severity) observed upon exposure to the antigen as compared with the symptoms observed when antigen is (or was) encountered without prior exposure to the desensitizing composition. As will be appreciated by those of ordinary skill in the art, the effective amount of blocking agent may vary depending on whether the desired biological endpoint is short-term desensitization (e.g., lasting up to 48 hours) or long term desensitization (e.g., lasting weeks, months, or years). The effective amount of antigen in a tolerizing composition is the amount that, when administered to an individual who is sensitized to an antigen, results in tolerization of the individual to the antigen. Achieving tolerance may require multiple administrations of the tolerizing composition. As will be appreciated by those of ordinary skill in the art, the effective amount of antigen in any single tolerizing administration is typically smaller than the amount of antigen that causes onset of severe allergic symptoms (e.g., anaphylaxis). On the other hand, higher concentrations of antigen may be employed if the individual has previously been exposed to an inventive desensitizing composition.

“Fragment”: An antigen “fragment” according to the present invention is any part or portion of the antigen that is smaller than the entire, intact antigen. In preferred embodiments of the invention, the antigen is a protein and the fragment is a peptide.

“IgE binding site”: An IgE binding site is a region of an antigen that is recognized by an anti-antigen IgE immunoglobulin. Such a region is necessary and/or sufficient to result in (i) binding of the antigen to IgE; (ii) cross-linking of anti-antigen IgE; (iii) degranulation of mast cells containing surface-bound anti-antigen IgE; and/or (iv) development of allergic signs and symptoms (e.g., histamine release). In general, IgE binding sites are defined for a particular antigen or antigen fragment by exposing that antigen or fragment to serum from allergic individuals. It will be recognized that different individuals may generate IgE that recognize different epitopes on the same antigen. Thus, it is typically desirable to expose antigen or fragment to a representative pool of serum samples. For example, where it is desired that sites recognized by human IgE be identified in a given antigen or fragment, serum is preferably pooled from at least 5-10, preferably at least 15, individuals with demonstrated allergy to the antigen. Those of ordinary skill in the art will be well aware of useful pooling strategies in other contexts.

“Immunodominant”: A particular epitope is considered to be “immunodominant” if it (i) is responsible for a significant fraction of the IgE binding observed with the intact antigen; (ii) is recognized by IgE in a significant fraction of sensitive individuals; and/or (iii) is a particularly high affinity site. An immunodominant epitope is often defined in reference to the other observed epitopes. For example, all IgE epitopes in a given antigen can be assayed simultaneously (e.g., by immunoblot), and the immunodominant epitopes can be identified by their strength as compared with the other epitopes. Usually, but not always, an immunodominant epitope will contribute at least 10% of the binding reactivity observed in such a study. Alternatively or additionally, an epitope can be classified as immunodominant if it is recognized by IgE in sera of a significant fraction, preferably at least a majority, more preferably at least about 60%, 70%, 80%, 90%, 95%, 99%, or 100%, of sensitive individuals.

“Inducing agents”: are compounds or other agents that induce a pAPC to produce stimulating cytokines. For example, if it is desired that a pAPC secrete Th1 stimulating cytokines, then factors such as LPS, CD40, CD40 ligand, BCGs, oligonucleotides containing CpG motifs, TNF, and microbial extracts such as preparations of Staphylococcus aureus, heat killed Listeria, modified cholera toxin, etc. can act as inducing agents (“Th1 inducing agents”). If instead it is desired that a pAPC secrete Th2 stimulating cytokines, then other factors (e.g., factors that induce IL-4 expression or inhibit IL-12 expression) can act as inducing agents (“Th2 inducing agents”).

“Mast cell”: As will be apparent from context, the term “mast cell” is often used herein to refer to one or more of mast cells, basophils, and other cells with IgE receptors.

“Offending IgE”: The term “offending IgE” refers to any IgE that is a part of an immunological response to an antigen. In a preferred embodiment, the offending IgE plays a role in the individual's allergic reaction to the antigen.

“Passive desensitization”: The term “passive desensitization” refers to a method of desensitizing an individual to at least one antigen. Without being bound by any particular theory, passive desensitization is thought to work by blocking the antigen binding sites of IgE molecules that are involved in the allergic response to the antigen. In other words, rather than being immunomodulatory, passive desensitization works by competing with the natural antigen for binding of the antigen-binding sites on IgE molecules. This theory does not rule out the possibility that the IgE-blocking agents may inherently lead to long term desensitization through immunomodulation (e.g., down-regulation of IgE production, down-regulation of Fc R1 receptor, down-regulation of Fc RII receptors, etc.).

“Peptide”: According to the present invention, a “peptide” comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofamesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyladenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Sensitized animal”: A “sensitized animal” is an animal having adapted an immunological state so that, when it encounters an antigen, it has a response similar to that observed in allergic humans. In one preferred embodiment, the initial reaction to the antigen consists primarily of cutaneous reactions with puffiness around the eyes and mouth, and/or diarrhea followed by a respiratory reaction such as wheezing and labored respiration. In another preferred embodiment, the percentage of degranulated mast cells is significantly higher in the sensitized animal versus the unsensitized animal. In yet another preferred embodiment, plasma histamine levels are significantly increased in the sensitized animal when it is challenged with antigen. In another preferred embodiment, there is an increased level of antigen specific IgG1 antibodies in the animal after sensitization. In a particularly preferred embodiment of the invention, the response is mediate by IgE immunoglobulin. In another preferred embodiment, the response is an anaphylactic reaction.

“Sensitized mast cell”: A “sensitized” mast cell is a mast cell that has surface-bound antigen specific IgE molecules. The term is necessarily antigen specific. That is, at any given time, a particular mast cell will be “sensitized” to certain antigens (those that are recognized by the IgE on its surface) but will not be sensitized to other antigens.

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“Susceptible individual”: According to the present invention, a person is susceptible to a severe and/or anaphylactic allergic reaction if (i) that person has ever displayed symptoms of allergy after exposure to a given antigen; (ii) members of that person's genetic family have displayed symptoms of allergy against the allergen, particularly if the allergy is known to have a genetic component; and/or (iii) antigen-specific IgE are found in the individual, whether in serum or on mast cells.

“Th1 response” and “Th2 response”: Th1 and Th2 responses are well-established alternative immune system responses that are characterized by the production of different collections of cytokines and/or cofactors. For example, Th1 responses are generally associated with the production of cytokines such as IL-1, IL-2, IL-12, IL-18, IFN, IFN, TNF, etc.; Th2 responses are generally associated with the production of cytokines such as IL-4, IL-5, IL-10, etc. The extent of T cell subset suppression or stimulation may be determined by any available means including, for example, intra-cytoplasmic cytokine determination. In preferred embodiments of the invention, Th2 suppression is assayed, for example, by quantitation of IL-4, IL-5, and/or IL-13 in stimulated T cell culture supernatant or assessment of T cell intra-cytoplasmic (e.g., by protein staining or analysis of mRNA) IL-4, IL-5, and/or IL-13; Th1 stimulation is assayed, for example, by quantitation of IFN, IFN, IL-2, IL-12, and/or IL-18 in activated T cell culture supernatant or assessment of intra-cytoplasmic levels of these cytokines.

### BRIEF DESCRIPTION OF THE DRAWING

FIG. 1 shows the ability of each peptide of Ara h 2 to stimulate T cells. Each peptide was tested, using standard techniques, on 19 different T cell preparations. Positive scores, defined as a T cell stimulation index of >2, are indicated by shading.

FIG. 2 shows the modified amino acid sequences of Ara h 1, Ara h 2, and Ara h 3. Altered positions are underlined.

FIG. 3 shows a decrease in Ara h 2-specific IgE in blood of mice desensitized with modified Ara h 2 protein.

FIG. 4 shows histamine levels after challenge in mice treated with a peptide mixture versus contols. Mice were challenged with native (N) Ara h 2 protein and reduced (R) Ara h 2 protein.

### DETAILED DESCRIPTION OF CERTAIN PREFERRED EMBODIMENTS OF THE INVENTION

The present invention provides a system for blocking/inhibiting an individual's allergic response to an antigen and a method of blocking/inhibiting and then administering the offending antigen in order to desensitize the individual to the offending antigen.

Individual

The individual treated in accordance with the present invention is allergic (i.e., exposure to the antigen causes an allergic reaction) to an antigen or at risk of becoming allergic to an antigen. The individual may be an animal or human. Preferred animals include, for example, pets (e.g., dogs, cats, ferrets, rabbits, birds, fish, reptiles, amphibians), farm animals (e.g., horses, cattle, goats, sheep, cows), and research animals (e.g., monkeys, apes, chimpanzees, mice, rats, rabbits). The individual preferably is a mammal and more preferably a human. The individual may be allergic to any antigen. Preferably, the antigen is an anaphylactic antigen, and more preferably, the antigen is a food antigen. In certain preferred embodiments, the antigen is an allergenic protein antigen. The individual's response to exposure to the offending antigen may range from mild to quite severe including anaphylaxis and death. Mild reactions include itching, watery eyes, hives, etc. More severe symptoms include wheezing, difficulty breathing, gastrointestinal disturbance, etc. The most severe allergic reaction is anaphylaxis and death.

IgE-Blocking Agents

The agents to be used to block the antigen binding sites of IgE molecules may comprise any available compound. For example, inventive blocking agents may comprise proteins, small molecules, peptides, antibodies, fragments of antibodies, polysaccharides, polynucleotides, etc. Multiple different compounds can be combined together to create a single blocking agent. In such circumstances, each individual compound is a blocking agent “component”. Blocking agent components are characterized by an ability to bind to anti-antigen IgE. Preferably, each component binds to only one IgE molecule at a time and cannot cross-link IgE molecules. Inventive blocking agents may indiscriminately block all IgE molecules that are present in an individual, but in most preferred embodiments, a given blocking agent will bind to only a subset of an individual's IgE (e.g., that corresponding to the collection of IgEs in the individual that recognize a particular antigen, family of antigens, or antigen epitope). In one preferred embodiment of the invention, the blocking agent only blocks the IgE molecules which bind a selected antigen to which the individual is to be exposed during immunotherapy.

Preferably, the kinetics of the interaction of the binding agent with IgE is such that the on rate is fast, preferably within hours and more preferably within minutes, and the off rate is slow, preferably within hours and more preferably within days, so that the blocking of the IgE will be effective for as long as possible. The agent may block the IgE from hours to weeks to months. Preferably, the agent blocks the IgE for a time period ranging from days to weeks. As will be appreciated by those skilled in this art, passive desensitization caused by the blocking agent composition may not persist beyond the lifetime of the blocking agent/IgE interaction (although the invention does not exclude the possibility that, in some circumstances, longer persistence may be achievable, for example if the blocking agent also includes, or is administered together with a composition that includes one or more compounds with immunomodulatory capabilities). Thus, if antigen is to be administered subsequent to blocking agent administration, it is generally preferred that such antigen administration be accomplished during the passive desensitization period. Furthermore, administered antigen should preferably be cleared from the individual's system before the passive desensitization period ends. In cases in which there is a risk that antigen may not be completely cleared before the end of the passive desensitization period (e.g., when the exact timing of antigen administration is unknown as might occur in accidental administration), additional administrations of blocking agent prior to the end of the passive desensitization period may be desirable.

In a particularly preferred embodiment of the inventive blocking agent, the blocking agent contains one or more components that bind to IgEs recognizing Immunodominant epitopes. Since most of the circulating and therefore mast-cell-bound IgE molecules are directed to such immunodominant epitopes, blocking of the antigen binding sites on these IgE molecules is expected to result in a substantial decrease in the severity of the allergic response upon exposure to the antigen. In other preferred embodiments, there may be more than one epitope which is particularly important in the allergic response so that the composition of blocking agents may contain blocking agents that compete for antigen binding to IgE molecules recognizing different epitopes.

As described herein, inventive blocking agents may contain components that block IgE directed against linear and/or conformational antigen epitopes.

In a preferred embodiment, the blocking agent comprises one or more peptides. For example, the agent may comprise peptides that are fragments of a polypeptide antigen, where each peptide of the agent possesses only one functional IgE binding site. This allows the peptide to block the antigen binding site of the IgE but does not allow the peptide to cross-link IgE on mast cells and thereby activate the mast cell. The design, identification, synthesis, and administration of such collections of peptides may be considered with reference to U.S. patent application (U.S. Ser. No. 09/455,294), filed Dec. 6, 1999, which is incorporated herein by reference in its entirety.

In another preferred embodiment, the agent comprises a mimeotope (i.e., a compound chemically unrelated to the antigen of interest) (see Example 6). A mimeotope may be a peptide, protein, a small molecule or other compound. Preferably, the mimeotope blocks antigen-specific IgE molecules. In a particularly preferred embodiment, a mimeotope is used to block IgE molecules directed against conformational epitopes of proteins or against non-polypeptide antigens. Methods of synthesizing or identifying mimetopes are well known to those skilled in the art.

In another preferred embodiment, the agent comprises an immunoglobulin or immunoglobulin fragment that does not cross-link IgE on mast cells. The immunoglobulin may be of any class such as IgE, IgG, IgM, IgA, etc. The immunoglobulin may also be altered. For example, the immunoglobulin may be humanized if it is to be administered to humans. In certain preferred embodiments, the immunoglobulin may bind and block all IgE molecules (Magnusson et al. Int. Arch. Allergy Appl. Immun. 80:329-332, 1986; incorporated herein by reference). In other preferred embodiments, the immunoglobulin may bind a subset of IgE such as those that bind a particular antigen (e.g., peanut antigen). In yet another preferred embodiment, the antibody is an anti-idiotype antibody. The blocking agent may also comprise a collection of antibodies selected to block various different antigen-binding sites on IgE molecules. In a preferred embodiment, the immunoglobulins are monoclonal antibodies (U.S. Pat. Nos. 4,491,632; 4,472,500; and 4,444,887; Methods in Enzymology 73B:3, 1981; each of which is incorporated herein by reference). Also preferred are single chain antibodies, antibody fragments (e.g., Fc fragments), etc.

In yet another preferred embodiment, the IgE-blocking agent comprises one or more small molecules. The small molecule may block the antigen binding sites of all IgE or only a subset of IgE such as those that bind a particular antigen. In a preferred embodiment, a set of IgE molecules is used to screen a library of chemical compounds. This library may be constructed using combinatorial chemistry (e.g., parallel or split-and-pool synthesis), or standard one-at-a-time synthetic techniques. The library may also be a collection of readily available compounds. A small molecule is chosen from the library based on its ability to bind the IgE without inducing cross-linking (see, for example, Example 6). Preferably, the binding affinity for the IgE is less than 1 M, more preferably less than 100 nM, and most preferably less than 10 nM.

In a particularly preferred embodiment, the IgE-blocking agent provides not only passive desensitization but also active desensitization (i.e., immunomodulation). The blocking agents while containing no more than one IgE binding site may contain T-cell epitopes that may lead to immunomodulation. In particularly preferred embodiments, the composition of IgE-blocking agents may contain peptides with no IgE-binding sites but with T-cell epitopes for the purpose of inducing a Th1 response or down-regulating the Th2 response.

Antigen

Any antigen to which an individual may be sensitive to is relevant to this invention. Preferred antigens include protein antigens, and of particular interest are anaphylactic protein antigens. Anaphylactic antigens include food antigens, insect antigens, and rubber antigens (e.g., latex). In particular, peanut and tree nut (e.g., walnut, almond, pecan, cashew, hazelnut, pistachio, pine nut, brazil nut) antigens, dairy (e.g., egg, milk) antigens, seed (e.g., sesame, poppy, mustard) antigens, fish/shellfish (e.g., cod, shrimp, crab, lobster, clams) antigens, and insect antigens are anaphylactic antigens according to the present invention. Particularly preferred anaphylactic antigens are food antigens; peanut (e.g., Ara h 1-3), milk, egg, and fish/shellfish (e.g., tropomyosin) antigens are especially preferred. In some cases, it will be desirable to work in systems in which a single compound (e.g., a single protein) is responsible for most observed allergies. In other cases, the invention can be applied to more complex allergens. In a particularly preferred embodiment, the antigen has predominately linear epitopes.

Environmental antigens may also be used in the present invention. Environmental antigens include animal dander, tree pollen, grass pollen, weed pollen, mites, dust mites, animal antigens, insect antigens, and fungal antigens. Specific examples of these antigens are listed in the Appendix of U.S. patent application (U.S. Ser. No. 09/455,294), filed Dec. 6, 1999 (incorporated in its entirety herein by reference).

Antigens for use in immunotherapy after passive desensitization by administering an IgE-blocking agent may be produced in any desired form. Preferably, the antigen is in a traditional immunotherapy formulation or a rush immunotherapy formulation. Such formulations are known in the art (Weber JAMA 278:1881-1887, 1997; Fornadley Otolaryngology Clinics North America 31:111 -127, 1998; Remington's Pharmaceutical Sciences 19th Ed., Mack Publishing, Chapter 82, 1995; each of which is incorporated herein by reference).

Crude antigen may be used, or antigen may be partially or completely pure. Protein antigens may be provided in their native form, in denatural form, and/or in recombinant form. In some cases, it will be desirable to administer to the individual an antigen composition that approximates as closely as possible the form of the antigen in nature. Such a formulation is not required, however.

In some embodiments, the antigen may be administered in association with cytokines, adjuvants, inducting agents, or other immunomodulatory substances. In some embodiments of the invention, a protein antigen is provided by a polynucleotide encoding the antigen. DNA or RNA may be used in the invention; however, DNA is generally preferred given its greater stability. The polynucleotide may be provided in the context of a delivery vector such as a plasmid or virus. Preferably, the polynucleotide includes expression sequences (e.g., promoter, enhancer, splicing signals, Shine-Delgarno sequence, etc.) sufficient to direct protein expression in the relevant individual. A wide variety of such sequences is known in the art (Sambrook et al. Molecular Cloning. A Laboratory Manual, 2nd Ed., 1989; Miller & Calos, eds., Gene Transfer vectors for Mammalian Cells, 1987; Ausubel et al., eds., Current Protocols in Molecular Biology, 1987; each of which is incorporated herein by reference). Once inside a cell, the polynucleotide is transcribed and translated in order to produce the protein antigen in situ. Production of the protein antigen leads to sensitization of the individual. In a preferred embodiment, the expression system (e.g., promoter, enhancer, splicing signals, etc.) and vector are matched to the species to which it is being administered. For example, if a mammal such as a mouse was to be treated using immunotherapy, the promoter used to drive protein production might be the cytomegalovirus (CMV) promoter.

Modified versions of the antigens may also be used in the present invention. Any type of modification can be used. They may be biological or chemical. The antigen may contain unnatural amino acids; may be modified, e.g., glycosylated, phosphorylated, hydroxylated, etc., may lack modification; may be cross-linked; may contain mutations (e.g., substitutions, deletions); etc. Antigens may also be a fusion protein (e.g., fused with a cytokine, another antigen, the same antigen, an inducing factor, an adjuvant, etc.)

Adjuvants

Compositions utilized in the practice of the present invention may include, or may be administered as part of a protocol that includes one or more adjuvants, cytokines, or inducing agents. Any adjuvant may be used in accordance with the present invention. A large number of adjuvant compounds is known; a useful compendium of many such compounds is prepared by the National Institutes of Health and can be found on the world wide web (http:/www.niaid.nih.gov/daids/vaccine/pdf/compendium.pdf, incorporated herein by reference; see also Allison Dev. Biol. Stand. 92:3-11, 1998; Unkeless et al. Annu. Rev. Immunol. 6:251-281, 1998; and Phillips et al. Vaccine 10:151-158,1992, each of which is incorporated herein by reference). Preferred adjuvants include those that suppress the Th2 response and/or enhance the Th1 response. Hundreds of different adjuvants are known in the art and could be employed in the practice of the present invention. Particularly preferred are those that induce IL-12 production, including microbial extracts such as fixed Staphylococcus aureus, Streptococcal preparations, Mycobacterium tuberculosis, lipopolysaccharide (LPS), oligonucleotides containing CpG motifs, Listeria monocytogenes, Toxoplasma gondii, Leishmania major, etc.

Cytokines and Inducing Agents

As mentioned above, inventive compositions may optionally be administered with at least one inducing agent or cytokine. The cytokine(s) or inducing agent(s) to be administered is/are preferably selected to reduce production of a Th2 and/or promote production of a Th1 response and thereby to reduce allergic symptoms, as discussed above. Cytokines that, when expressed during antigen presentation to a T cell, induce a Th1 response in T cells (i.e., “Th1 stimulating cytokines”) include IL-12, IL-2, IL-18, IL-1 or fragments thereof, IFN, and/or IPN, etc.; Th2 stimulating cytokines include IL-4. Inducing agents that prompt the expression of Th1 stimulating cytokines include factors such as LPS, BCGs, CD40, CD40 ligand, oligonucleotides containing CpG motifs, TNF, and microbial extracts such as preparations of Staphylococcus aureus, heat killed Listeria, search specs delete any time it appears, etc.; inducing agents that prompt the expression of Th2 stimulating cytokines include agents that induce IL-4 expression by T cells or other cells, as well as agents that suppress IL-12 expression by pAPC.

In a particularly preferred embodiment of the present invention, the inducing agent is a peptide derived from the antigen to which the individual is allergic. Preferably, the peptide has either one or zero intact IgE binding sites and has at least one T-cell epitope. These peptides preferably promote a Th1 response and/or reduce the Th2 response.

Cytokines or inducing agents may be provided as impure preparations (e.g., isolates of cells expressing a cytokine gene, either endogenous or exogenous to the cell), but are preferably provided in purified form. Purified preparations are preferably at least about 90% pure, more preferably at least about 95% pure, and most preferably at least about 99% pure. Alternatively, genes encoding the cytokines or inducing agents may be provided, so that gene expression results in cytokine or inducing agent production either in the individual being treated or in another expression system (e.g., an in vitro transcription/translation system or a host cell) from which expressed cytokine or inducing agent can be obtained for administration to the individual.

Where both cytokine/inducing agent/adjuvant and desensitizing composition/tolerizing composition are to be administered to an individual, they may be provided together or separately. For example, both may be associated by means of a common encapsulation device or by means of physical association such as covalent linkage, hydrogen bonding, hydrophobic interaction, electrostatic interactions, van der Waals interaction, etc. In certain preferred embodiments of the invention in which the compounds to be administered are polypeptides that are to be provided together, genes encoding the polypeptides may be provided. In such circumstances, genes for two or more polypeptides may be provided as part of the same nucleic acid molecule, or each may be provided as a separate nucleic acid. In some embodiments, two or more factors may be expressed from a single gene, as a fusion protein. Alternatively or additionally, multiple genes may be linked to the same or equivalent control sequences, so that they become expressed within the individual in response to the same stimuli. A wide variety of different control sequences, active in different host cells under different conditions, is available in the art. Any such control sequences, including constitutive control sequences, inducible control sequences, and repressible control sequences, may be used in accordance with the present invention, though inducible or repressible sequences are particularly preferred for applications in which additional control over the timing of gene expression is desired.

Coordinate control is particularly desirable where one or more of the cytokines, inducing agents, adjuvants, blocking agents, or antigens being employed is a heterodimeric compound (e.g., IL-12). In such cases, it will generally be desirable to express both dimmer components at comparable levels, preferably under control of the same regulatory elements. Also, fusions may be made with one or both dimer components.

Administration

Those of ordinary skill in the art will appreciate that compositions to be administered to individuals according to the present invention may be administered via any of a variety of routes, protocols, and dosing regimens. Known routes of administration include, for example, intravenous (IV), intraperitoneal (IP), intragastric (IG), subcutaneous (SQ), intramuscular (IM), oral (PO), rectal (PR), intrathecal, vaginal, intranasal, transdermal, intradermal, etc. Intravenous, intramuscular, transdermal, intradermal, intranasal, and oral deliveries are generally preferred.

As discussed above, according to the present invention, a composition comprising one or more blocking agents is administered to an individual who is sensitive to a particular antigen, so that the individual's sensitivity to that antigen is reduced for a period of time. Although not wishing to be bound by any particular theory, we refer to this process herein as “passive desensitization” (as distinguished from immunomodulation) because no active alteration of the individual's immune reaction is required. Rather, so long as the blocking agent is able to biochemically compete with the antigen for binding to anti-antigen IgE, the individual's sensitivity reaction to the antigen should be reduced. Of course, immunomodulation or other effects are not precluded from the present invention. Embodiments of the invention that utilize adjuvants, cytokines, and/or inducing agents are often expected to involve immunomodulation.

Those of ordinary skill in the art, given the teachings provided herein, will readily be able to determine a desirable dosing regimen for administration of a particular blocking agent to a particular individual. For example, if short-term desensitization to a particular antigen is required (e.g., so that rush immunotherapy with that antigen can be performed or because the individual will face a short term high risk of encountering the antigen), one or a small number (fewer than 10, preferably fewer than 5, more preferably fewer than 3) of administrations may be appropriate. On the other hand, if longer-term (e.g., for a period of days, weeks, months, or years) desensitization is desired, repeated administrations over the longer-term period of time may be appropriate.

Administrations of the inventive blocking composition may often be followed by administrations of antigen, either accidentally or intentionally. Intentional antigen administrations include immunotherapeutic administrations (e.g., ones intended to reduce the individual's undesirable immunologic response to the antigen). Any protocol that achieves sufficiently long term antigen tolerance is appropriate in such circumstances. For many antigens, established protocols for either standard or rush immunotherapy are available (Weber JAMA 278:1881-1887, 1997; Stevens Acta Clinica Beligica 53:66-72, 1998; Canadian Society of Allergy and Clinical Immunology Can. Med. Assoc. J. 152:1413-1419, 1995; Fomadley Otolaryngology Clinics North America 31:111-127, 1998; Remington 's Pharmaceutical Sciences 19th Ed., Mack Publishing, 1995, Chapt 82; each of which is incorporated herein by reference). Dosages for each antigen depend upon the particular antigen as well as the individual receiving the therapy, and further are to some extent at the discretion of the individual practitioner. Those of ordinary skill in the art are fully able to determine the proper dose. Generally, standard immunotherapy involves weekly administration of increasing doses (in the microgram range) of antigen. The amount of antigen is increased until alleviation (to elimination) of symptoms is observed or adverse symptoms are observed. Rush immunotherapy, by contrast, involves initial clustered administrations of antigen (such as several in a day, once a week), followed by weekly administration. The therapy is typically given repeatedly for as long as symptoms exist, with a maintenance dose given at two to four week intervals. Over time, maintenance doses may be scheduled even less frequently, such as every six to eight weeks.

In general, the amount of IgE-blocking-agent composition or antigen composition administered per dose may range from, for example, 0.0001 mg/g body mass to 10 mg/g body mass. Preferably, the dosage used ranges from 0.001 mg/g body mass to 1 mg/g body mass. More preferably, the dosage used ranges from 0.001 mg/g body mass to 0.1 mg/g body mass. Those of ordinary skill in the art will be aware of techniques, including those described herein, for delivering the optimum desensitizing dose and protocol. For example, a known amount of an IgE-blocking agent may be administered, followed by measurement of the unblocked antigen-specific IgE in sera, assessment of hypersensitivity responses (e.g., skin test to the relevant antigen), detection of vascular leakage, determination of plasma histamine levels, testing for passive cutaneous anaphylaxis, quantitation of cytokine proteins, examination of histology of mast cells, and/or determination of serum antigen concentration.

Where administration of a composition according to the present invention involves administration of one or more nucleic acids, the preferred amount of DNA per dose typically ranges from about 0.01 g to 1 mg if the nucleic acid is DNA. Preferably, the amount of DNA per dose is about 0.1 g to 100 g DNA; and more preferably, the amount of DNA per dose is about 1 g to 50 g DNA. If RNA is to be used as the encoding nucleic acid, more RNA may need to be administered due to the short half-life of RNA in vivo.

The immunotherapy may employ a combination of antigen per se and DNA encoding the antigen. They may be administered at the same time or separately.

The IgE-blocking agent composition and the antigen composition may be formulated as one or more pharmaceutical compositions, as discussed below. Also, one or more adjuvants, cytokines, or inducing agents may be included in the blocking agent composition, or, more preferably, the antigen composition. Alternatively or additionally, adjuvant, cytokine, and/or inducing agent may be provided in one or more separate compositions.

Pharmaceutical Compositions

Pharmaceutical compositions for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of an agent, it is often desirable to slow the absorption of the drug from subcutaneous or intramuscular injection. This may be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of agent to polymer and the nature of the particular polymer employed, the rate of release of the agent can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The compounds can also be in micro-encapsulated form with one or more excipients as noted above. The solid dosage forms of tablets, dragees, capsules, pills and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compounds of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Mapping IgE Binding Sites in Peanut Antigens

Introduction

This Example describes the definition and analysis of IgE binding sites within peanut protein antigens. The information presented may be utilized in accordance with the present invention, for example, to prepare one or more peptides, or collections thereof, each peptide containing only one peanut antigen IgE binding site. In general, any of a variety of methods (e.g., immunoprecipitation, immunoblotting, cross-linking, etc.) can be used to map IgE binding sites in antigens (see, for example, methods described in Coligan et al. (eds.) Current Protocols in Immunology, Wiley & Sons, and references cited therein, incorporated herein by reference). Generally, an antigen or antigen fragment (prepared by any available means such as, for example, chemical synthesis, chemical or enzymatic cleavage, etc.) is contacted with serum from one or more individuals known to have mounted an immune response against the antigen. Where the goal is to map all observed IgE binding sites, it is desirable to contact the antigen or antigen fragment, simultaneously or serially, with sera from several different individuals since different epitopes may be recognized in different individuals. Also, different individuals may react differently to the same antigen or antigen fragments; in certain circumstances it may be desirable to map the different IgE binding sites observed in different individuals.

It will be appreciated that an IgE binding site that is strongly recognized in the context of an intact antigen may not be strongly bound in an antigen fragment even though that fragment includes the region of the antigen corresponding to the binding site. As will be clear from context, in some circumstances an antigen fragment is considered to contain an IgE binding site whenever it includes the region corresponding to an IgE binding site in the intact antigen; in other circumstances, an antigen fragment is only considered to have such a binding site if physical interaction has actually been demonstrated as described herein.

Materials and Methods

IGE IMMUNOBLOT ANALYSIS: Membranes to be blotted were prepared either by SDS-PAGE (performed by the method of Laemmli Nature 227:680-685, 1970; incorporated herein by reference) of digested peanut antigen or by synthesis of antigen peptides on a derivativized cellulose membrane. SDS-PAGE gels were composed of 10% acrylamide resolving gel and 4% acrylamide stacking gel. Electrophoretic transfer and immunoblotting on nitrocellulose paper was performed by the procedures of Towbin (Proc. Natl. Acad. Sci. USA 76:4350-4354, 1979; incorporated herein by reference).

For mapping of human IgE binding sites, the blots were incubated with antibodies (serum IgE) from 15-18 patients with documented peanut hypersensitivity. Each of the individuals had a positive immediate skin prick test to peanut and either a positive, double-blind, placebo-controlled food challenge or a convincing history of peanut anaphylaxis (laryngeal edema, severe wheezing, and/or hypotension). At least 5 ml of venous blood was drawn from each patient and allowed to clot, and the serum was collected. All studies were approved by the Human Use Advisory Committee at the University of Arkansas for Medical Sciences. Serum was diluted in a solution containing TBS and 1% bovine serum albumin and incubated with antigen for at least 12 h at 4° C. or for 2 h at room temperature. The bound primary antibody was detected with ¹²⁵I-labeled anti-IgE antibody (Sanofi Diagnostics Pasteur Inc., Paris, France).

For mapping of murine IgE binding sites, a blot containing overlapping 13 mer peptides, offset by 2 amino acids, was incubated with serum from peanut sensitized mice as described in U.S. patent application Ser. No. 09/455,294, filed Dec. 6, 1999; incorporated herein by reference.

PEPTIDE SYNTHESIS: Individual peptides were synthesized on a derivativized cellulose membrane using Fmoc amino acid active esters according to the manufacturer's instructions (Genosys Biotechnologies, Woodlands, Tex.). Fmoc-amino acid derivatives were dissolved in 1-methyl-2-pyrrolidone and loaded on marked spots on the membrane. Coupling reactions were followed by acetylation with a solution of 4% (v/v) acetic anhydride in N,N-dimethyl formamide (DMF). After acetylation, Fmoc groups were removed by incubation of the membrane in 20% (v/v) piperdine in DMF. The membrane was then stained with bromophenol blue to identify the location of the free amino groups. Cycles of coupling, blocking, and deprotection were repeated until the peptides of the desired length were synthesized. After addition of the last amino acid in the peptide, the amino acid side chains were deprotected using a solution of dichloromethane/trifluoroacetic acid/triusobutylsilante (1/10/0.05). Membranes were either probed immediately or stored at −20° C. until needed.

Results

Human IgE binding sites have previously been mapped for Ara h 1 (Burks et al., J. Clin. Invest. 96:1715-1721, 1995; U.S. Ser. No. 90/141,220, filed Aug. 27, 1998; each of which is incorporated herein by reference) and Ara h 2 (Stanley et al., Arch. Biochem. Biophys. 342:244-253, 1997; U.S. Ser. No. 90/141,220, filed Aug. 27, 1998; each of which is incorporated herein by reference). We have also mapped such epitopes for Ara h 3 (Rabjohn et al., J. Clin. Invest. 103:535-542, 1999; U.S. Ser. No. 90/141,220, filed Aug. 27, 1998; each of which is incorporated herein by reference). We have also mapped murine IgE binding sites for Ara h 2, by probing filters containing overlapping 20 mers, offset by 5 amino acids, that span the Ara h 2 sequence with serum from mice sensitized to recombinant Ara h 2.

The results of these studies are summarized below in Tables (essential residues are underlined, immunodominant epitopes are in bold).

TABLE 1
Human IgE Binding Epitopes in Ara h 1

| EPITOPE NUMBER | SEQUENCE | POSITION |
|---|---|---|
| 1 | AKSSPYOKKT | 25-34 |
| 2 | OEPDDLKOKA | 48-57 |
| 3 | LEYDPRLVYD | 65-74 |
| 4 | GERTRGRQPG | 89-98 |
| 5 | PGDYDDDRRQ | 97-106 |
| 6 | PRREEGGRWG | 107-116 |
| 7 | REREEDWRQP | 123-132 |
| 8 | EDWRRPSHQQ | 134-143 |
| 9 | QPRKIRPEGR | 143-152 |
| 10 | TPGOFEDFFP | 294-303 |
| 11 | SYLQEFSRNT | 311-320 |
| 12 | FNAEFNEIRR | 325-334 |
| 13 | EQEERGORRW | 344-353 |
| 14 | DITNPINLRE | 393-402 |
| 15 | NNFGKLFEVK | 409-418 |
| 16 | GTGNLELVAV | 461-470 |
| 17 | RRYTARLKEG | 498-507 |
| 18 | ELHLLGFGTN | 525-534 |
| 19 | HRIFLAGDKD | 539-548 |
| 20 | IDOIEKOARD | 551-560 |
| 21 | KDLAFPGSGE | 559-568 |
| 22 | KESHFVSARP | 578-587 |
| 23 | PEKESPEKED | 597-606 |

TABLE 2
Human and Mouse IgE Binding Epitopes in Ara h 2

| SEQUENCE OF HUMAN | SEQUENCE OF MOUSE EPITOPE | |
|---|---|---|
| EPITOPE (NUMBER) | (NUMBER) | POSITION |
| HASARQQWEL (1) | LFLLAAH (1) | H15-24 |
| | | M9-15 |
| QWELQGDRRC (2) | RQQWELQGDRR (2) | H21-28 |
| | | M19-29 |
| DRRCOSOLER (3) | RCQSQLERA (3) | H27-36 |
| | | M29-37 |
| LRPCEQHLMQ (4) | | H39-48 |
| KIQRDEDSYE (5) | DEDSYERDP (4) | H49-56 |
| | | M53-61 |
| YERDPYSPSQ (6) | YERDPYSPS (5) | H59-64 |
| | | M57-65 |
| SQDPYSPSPY (7) | YSPSPYD (6) | H65-72 |
| | | M69-75 |
| DRLOGRQQEQ (8) | QQEQQFK (7) | H117-122 |
| | | M121-127 |
| KRELRNLPQQ (9) | KIRELRNLPQ (8) | H127-132 |
| | | M127-135 |
| | RNLPQQCGL (9) | M131-139 |
| | CGLRAPQ (10) | M137-143 |
| QRCDLDVESG (10) | QRCDLDV (11) | H143-152 |
| | | M143-149 |

Human Ara h 2 epitopes (6) and (7), and mouse Ara h 2 epitopes (5) and (6) were considered to be immunodominant because, in each case, the two epitopes combined contributed about 40-50% of the observed IgE reactivity (as determined by densitometric analysis of the blot). Mouse epitope (3) was also considered to be immunodominant, as it contributed as much as about 15% of the IgE reactivity. No other mouse or human epitope contributed more than about 10% of the reactivity.

TABLE 3
Human IgE Binding Epitopes in Ara h 3

| EPITOPE NUMBER (FRACTION | | |
|---|---|---|
| OF PATIENTS WITH IGE THAT | | POSI- |
| BIND) | SEQUENCE | TION |
| 1 (25%) | IETWNPNNQEFECAG | 33-47 |
| 2 (38%) | GNIFSGFTPEFLEQA | 240-254 |
| 3 (100%) | VTVRGGLRTLSPDRK | 279-293 |
| 4 (38%) | DEDEYEYDEEDRRRG | 303-317 |

Epitope 3 of Ara h 3 was designated as immunodominant because it was recognized by IgE in sera from all 10 patients tested.

### Example 2

### Collections of Ara h 2 Peptides

5/20 Native

A collection of 28 peptides, each 20 amino acids long and staggered by 5 amino acids, spanning the sequence of the native Ara h 2 protein (SEQ ID NO:2) was prepared as described above. Table 4 presents the sequences of the individual peptides:

TABLE 4
5/20 Native Ara h 2 Peptides

| PEPTIDE NO | SEQ ID NO: | SEQUENCE |
|---|---|---|
| 1 | | LTILVALALFLLAAHASARQ |
| 2 | | ALALFLLAAHASARQQWELQ |
| 3 | | LLAAHASARQQWELQGDRRC |
| 4 | | ASARQQWELQGDRRCQSQLE |
| 5 | | QWELQGDRRCQSQLERANLR |
| 6 | | GDRRCQSQLERANLRPCEQH |
| 7 | | QSQLERANLRPCEQHLMQKI |
| 8 | | RANLRPCEQHLMQKIQRDED |
| 9 | | PCEQHLMQKIQRDEDSYERD |
| 10 | | LMQKIQRDEDSYERDPYSPS |
| 11 | | QRDEDSYERDPYSPSQDPYS |
| 12 | | SYERDPYSPSQDPYSPSPYD |
| 13 | | PYSPSQDPYSPSPYDRRGAG |
| 14 | | QDPYSPSPYDRRGAGSSQHQ |
| 15 | | PSPYDRRGAGSSQHQERCCN |
| 16 | | RRGAGSSQHQERCCNELNEF |
| 17 | | SSQHQERCCNELNEFENNQR |
| 18 | | ERCCNELNEFENNQRCMCEA |
| 19 | | ELNEFENNQRCMCEALQQIM |
| 20 | | ENNQRCMCEALQQIMENQSD |
| 21 | | CMCEALQQIMENQSDRLQGR |
| 22 | | LQQIMENQSDRLQGRQQEQQ |
| 23 | | ENQSDRLQGRQQEQQFKREL |
| 24 | | RLQGRQQEQQFKRELRNLPQ |
| 25 | | QQEQQFKRELRNLPQQCGLR |
| 26 | | FKRELRNLPQQCGLRAPQRC |
| 27 | | RNLPQQCGLRAPQRCDLDVE |
| 28 | | QCGLRAPQRCDLDVESGGRD |

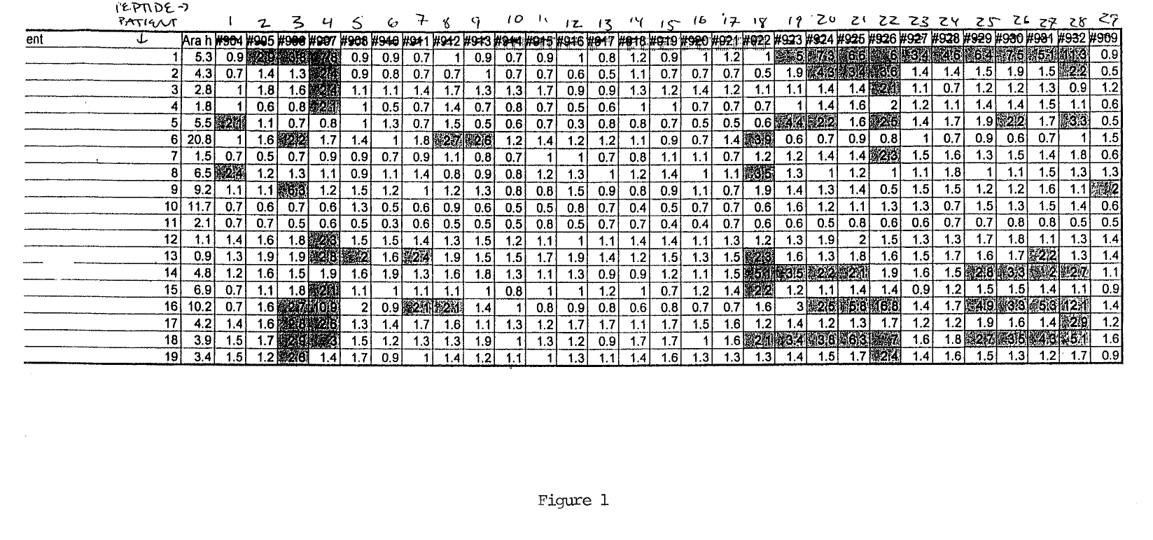

Each of these peptides was tested for its ability to stimulate T cells. The results are shown in FIG. 1. Each peptide was tested, using standard techniques, on 19 different T cell preparations. Positive scores, defined as a T cell stimulation index of >2, are indicated by shading. As can be seen, peptides 1-9 (especially 3 and 4) and 18-29 (especially 18-22 and 25-28) have significant T cell stimulation capability; peptides, 10-17 do not show such activity.

5/20 Native, Depleted for ≧2 Human Sites

In order to avoid crosslinking of IgE on mast cell surfaces, it is desirable to remove from a collection of Ara h 2 peptides those peptides that include two or more IgE binding sites, and therefore have the ability to cross-link anti-Ara h 2 IgE molecules. Individual peptides could be tested for their ability to simultaneously bind to two or more IgE molecules or could be identified by direct testing of IgE binding and/or cross-linking (e.g., histamine release). However, in the present Example, we simply designate those peptides that contain two complete IgE binding sites as determined by sequence analysis alone, relying on the above-described analyses to define the IgE binding sites. Under this analysis, peptides 3, 5, and 12 from Table 4 should be removed from the collection.

5/20 Single IgE Sites Only

One particularly preferred collection of Ara h 2 peptides contains only those peptides whose sequence includes only a single intact IgE binding site. For example, since Table 2 displays the Ara h 2 IgE binding sites and Table 4 gives the sequences of example 20-mer Ara h 2 peptides, it can readily be seen that peptides 2 and 3 contain human IgE epitope (1); peptides 3 and 4 contain human IgE epitope (2); peptides 5 and 6 contain human IgE epitope (3); peptides 7 and 8 contain human IgE epitope (4); peptides 9 and 10 contain human IgE epitope (5); peptides 10, 11, and 12 contain human IgE epitope (6); peptides 12 and 13 contain human IgE epitope (7); peptides 22, 23, and 24 contain human IgE epitope (8); peptides 24, 25, and 26 contain human IgE epitope (9); and peptides 27 and 28 contain human IgE epitope (10). Since those peptides that contain two intact IgE epitopes are preferably excluded from inventive blocking agent compositions, a preferred composition would include only peptides 2, 4, 5 and/or 6, 7 and/or 8, 9, 11, 13, 22 and/or 23, 25 and/or 26, and 27 and/or 28.

Alternatively, some preferred compositions might contain only those peptides whose sequence includes an immunodominant IgE epitope. For example, human epitopes (6) and (7) have been found to be immunodominant. It is possible that a composition containing only peptides 11 and 13 would be sufficient to block crosslinking of antibodies reactive against those epitopes. If such antibodies represent a sufficiently large percentage of the anti-Ara h 2 antibodies in an individual, then a significant reduction in their crosslinking could have a global protective effect against Ara h 2 sensitivity.

Those of ordinary skill in the art will readily appreciate that the design of Ara h 2 peptides containing one and only one IgE binding site is by no means limited to peptides having the precise lengths and sequences set forth in Table 2. In general, any peptide that contains a functional IgE binding site (as may be defined using any assay described herein or others known in the art) may be employed. It may often be desirable to select the peptide sequence so that the IgE binding site is approximately centered in the peptide. Also, it may be desirable to make modifications to the peptide to increase or alter solubility in a desired solvent. Additionally, there is no need for all of the peptides in a blocking agent composition to be of the same length.

Those of ordinary skill in the art will also appreciate that precisely the same principles that are described above for the selection of Ara h 2 blocking agent peptides can be applied to any polypeptide antigen. In general, (i) IgE binding sites present in the sensitive population are determined; (ii) peptide sequences containing one and only one IgE binding site are selected; and (iii) peptides having the selected sequences are obtained. Immunodominant epitopes may be selected if desired, possibly to the exclusion of less prevalent epitopes.

As discussed herein, it may sometimes be desirable to prepare blocking agent compositions comprising antigen fragments (e.g., IgE binding site and one or more T cell epitopes). In an analysis of 15 patients, we have found that at least Ara h 2 peptides 1, 2, 3, 8, 9 and 10 have both a single IgE site and a T cell epitope recognized by at least one person.

### Example 3

### Desensitization of PN-Sensitized Mice Using Ara h 2 Peptides

Introduction

This Example describes the use of a collection of antigen fragments (of the Ara h 2 protein) to desensitize individuals to peanut allergy. The Example also shows desensitization using a modified Ara h 2 protein whose IgE binding sites have been disrupted. The results with modified protein antigen are readily generalizable to peptide fragments, as described herein.

Materials and Methods

MICE AND REAGENTS: Female C3H/HeJ mice, 5-6 weeks of age were purchased from the Jackson Laboratory (Bar Harbor, Me.) and maintained on peanut (PN)-free chow, under specific pathogen-free conditions. Standard guidelines for the care and use of animals was followed.

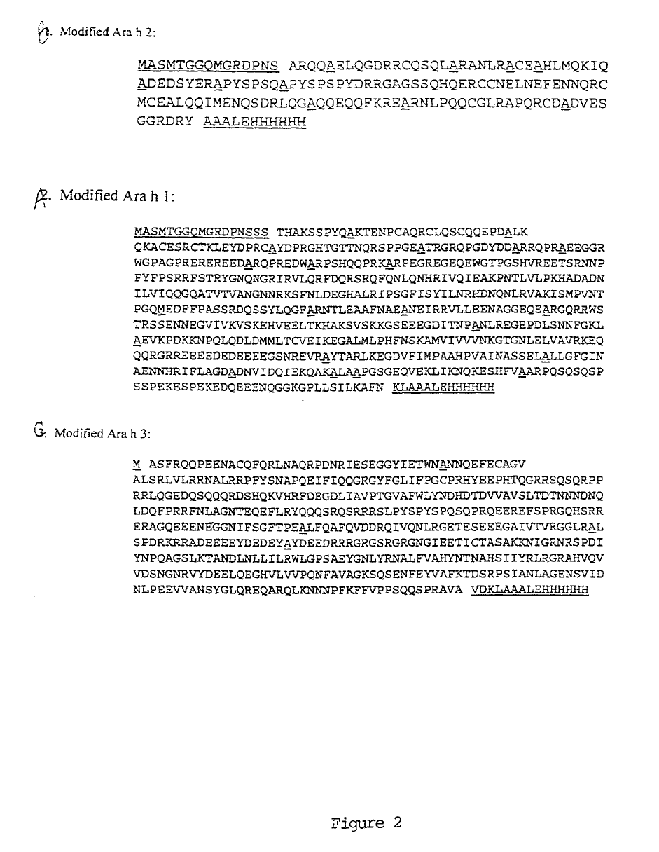

Ara h 2 protein was purified as described by Burks et al. (J. Allergy Clin. Immunol. 8:172-179, 1992; incorporated herein by reference). Modified Ara h 2 was prepared as described in U.S. Ser. No. 09/141,220, filed Aug. 27, 1998, incorporated herein by reference. The sequence of the modified Ara h 2 differed from that of natural Ara h 2 as indicated in FIG. 2 (altered positions are underlined). The Ara h 2 peptide collection was the 5/20 collection described above in Example 2.

SENSITIZATION: Mice were sensitized by ig feeding with 5 mg of Ara h 2 plus 0.3 g/gm body weight of cholera toxin (CT) as an adjuvant and were boosted twice, at weeks 1 and 3. Intragastric feeding was performed by means of a stainless steel blunt feeding needle as described by Li et al., J. Allergy Clin. Immunol. 103:206, 1999, incorporated herein by reference). Control mice received either CT alone or sham treatment.

DESENSITIZATION: Two weeks after sensitization, mice were treated with intranasal or subcutaneous peptide mix (either 2 g or 20 g), or with intranasal modified Ara h 2 (2 g). One set of control mice was treated with intranasal wild type Ara h 2; another set was mock treated.

CHALLENGE: Two weeks later, desensitized mice were challenged orally with 5 mg of wild type Ara h 2, divided into two doses of 2.5 mg 30 min apart.

ASSAYS: Hypersensitivity testing and IgE measurement were performed as described above in U.S. patent application Ser. No. 09/455,294, filed Dec. 6, 1999. Plasma histamine levels and airway responsiveness were also assayed, as were Ara h 2-specific IgE and IgG2 levels.

RECHALLENGE: The mice that were sensitized, desensitized, and challenged as described above in Example were rechallenged with Ara h 2 protein 3 weeks later.

Results

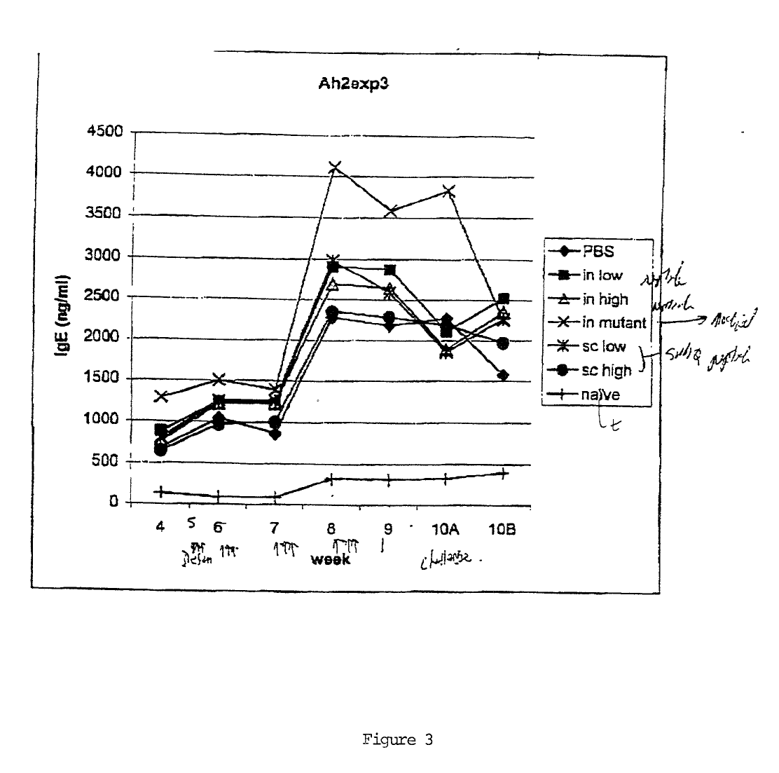

As shown in FIG. 3, anti-Ara h 2 IgE levels in mice exposed to native Ara h 2 rose four fold during the “desensitization period”. By contrast, these IgE levels did not increase significantly in mice exposed to low or high dose peptides, and actually decreased almost two-fold in mice exposed to modified Ara h 2. Moreover, significant protection from anaphylaxis was observed with both the high dose peptides and the modified protein. In order to determine whether this protection was long term, we rechallenged the mice several (three) weeks later. As shown below in Table 5, the observed protection was long term:

TABLE 5

| | ANTI ARA H 1 | SEVERITY OF |
|---|---|---|
| | IGE LEVELS | ANAPHYLACTIC |
| | DURING 3 WEEKS | SYMPTOMS AS |
| | BETWEEN | COMPARED WITH |
| “VACCINE” | CHALLENGES | FIRST CHALLENGE |
| sham | increased | worse |
| low [native peptides] | increased | worse |
| 20 mers, 5aa stagger | | |
| 2 g/mouse | | |
| high [native peptides] | no increase | no change |
| 20 mers, 5aa stagger | | |
| 20 g/mouse | | |
| modified protein | modest increase | no significant change |

These results clearly demonstrate that a collection of Ara h 2 peptides containing substantially all of the structural features of Ara h 2, can desensitize individuals allergic to Ara h 2.

### Example 4

### Passive Desensitization of Human Basophils

Introduction

One of the ways in which inventive blocking agents, or compositions containing them, may be characterized is by their ability to inhibit histamine release in isolated basophils that are contacted with antigen. The present Example describes one procedure by which such basophil histamine release is assayed; those of ordinary skill in the art will recognize that various modifications and alterations of this precise procedure can be made without departing from the spirit or scope of the present invention. Basophil histamine release assays are well established in the art (to give but a few examples, see Counsell et al., J. Allergy Clin. Immunol. 98:884, 1996; Haselden et al., J. Exp. Med. 189:1885, 1999; each of which is incorporated herein by reference).

Materials and Methods

REAGENTS: EDTA, 0.1M: 37.23 g Disodium EDTA; 600 ml H₂O; adjust pH to 7.18-7.20 with 50% NaOH; add H₂O to 1 L. 10×HBS: 80.0 g NaCl; 3.7 g KCl; 23.8 g HEPES (free acid); add H₂O to 1 L. Filter, autoclave, store at 4° C. 1×HBS, PH 7.4: Dilute 10×stock to 1×with distilled H₂O. Adjust pH with 10 N NaOH. Filter, autoclave, store at 4° C. HBS (+ALBUMIN): 100 ml 1×HBS; 0.125 ml 25% solution of Human serum albumin (ALBUMAC™ Baxter Scientific). HBS+1 mM CaCl₂: 100 ml HBS (+albumin); 10 μl CaCl₂; 50 μl MgCl₂

PROTOCOL: 15 ml of venous blood from a sensitized individual is drawn into a plastic syringe containing 5 ml of 0.1 M EDTA pH 7.2. Blood should be drawn gently to avoid lysis. Samples are transferred to 50 ml polycarbonate tubes containing 10 ml clinical Dextran 70. Preferably, the tubes have previously been washed without detergent and rinsed at least three times in distilled, deionized water. Blood should be poured down the side of the tube to avoid bubbles, and should be mixed by gentle swirling.

Cells are allowed to sediment at room temperature until a sharp interface develops between the red cells and plasma (which contains leukocytes and platelets), generally 60-90 mins. Cells should not sit longer than 2 hours. Plasma (buffy coat) layer is drawn off using a 3 ml plastic transfer pipette and is transferred to polycarbonate centrifuge tubes. Plasma is then centrifuged for 10 min at 450 g, 4° C.

Supernatant is carefully poured off, and the cell button is resuspended by gently shaking the tubes. Bubbles should be avoided. 30 ml (approximately 2×the initial cell volume) of cold HBS-albumin is added to the cells, the cells are resuspended and are recentrifuged for 10 min at 200 g, 4° C. This step, which functions as a wash, is repeated promptly. The supernatant is them completely poured off, and the cells are resuspended in 1 ml of cold HBS+3 mM CaCl₂. Cells are then counted and additional buffer is added to adjust the cell concentration to 1×10⁷ cells/ml. At this point, the cells are ready for use in basophil (leukocyte) histamine release assay.

For the histamine release assays, which should always be performed at least in duplicate, reaction tubes are prepared as follows: 50 l cold HBS+3 mM CaCl₂ are added to control (total and spontaneous release) tubes; 25 l cold HBS+3 mM CaCl₂ and 25 l HRF supernatant are added to test tubes. Two batches of test tubes are prepared: those that will receive cells that were not previously incubated with blocking agent, and those that will receive cells that were previously incubated with blocking agent (optionally, additional batches of test tubes are prepared that will receive cells that have been incubated with different concentrations of blocking agent).

Leukocyte suspension (in HBS+3 mM CaCl₂) is warmed at 37° C. for 6 min. In different tubes, leukocytes are mock incubated or are actually incubated with blocking agent for 15-120, preferably 15-30 minutes. Then, 50 l of the appropriate suspension is added to each reaction tube. Liquids are mixed by light finger vortexing. Tubes are then placed in a 37° C. bath for 45 minutes.

After the 45 minute incubation, total release control tubes are placed in boiling water for 10 minutes and then are immediately transferred to ice. 700 l of cold HBS+1 mM CaCl₂ are then added, and the tubes are kept on ice until other tubes have been processed. Challenge antigen is then added to test tubes. For example, challenge antigen may be in any desired form (e.g., crude, purified, recombinant, etc.). In certain situations, it may be desirable to use a form of the antigen that includes only a subset of all possible epitopes found in the antigen in its native form. For example, some epitopes may have minimal clinical relevance (e.g., if they are not normally encountered by sensitive individuals in the routes of exposure through which those individuals typically encounter antigen). Without wishing to be bound by any particular theory, we propose that conformational epitopes are not always clinically relevant for antigens that are naturally encountered orally because such epitopes often do not pass through the gastrointestinal lining. Thus, it may sometimes be desirable to exclude conformational epitopes (e.g., by using denatured or fragmented antigen) from the challenge antigen in order to minimize “false” (i.e., not clinically relevant) histamine release.

Test tubes and spontaneous release control tubes are processed by adding 700 l cold HBS+1 mm CaCl₂ to each tube and centrifuging the tubes immediately in a microcentrifuge. 750-800 l of supernatant is then transferred to a fresh tube. At this point, supernatants may be frozen or alternatively may be assayed for histamine content by spectrofluorimetry. Percentage of histamine release is calculated from the mean histamine release (ng/ml) values using the equation: % release=[(test sample)−(spontaneous)/(total release)−(spontaneous)]×100.

Results

Blocking agents are characterized as effective according to the present invention if the amount of histamine released from cells incubated with the blocking agent is reduced at least about 20%, preferably at least about 30%, 40%, 50%, 60%, 70%, 80%, 90%, 95%, 97%, 98% or 99% as compared with the amount of histamine released from corresponding cells that were not so incubated.

### Example 5

### Characterization of Blocking Agent Compositions

Competetive ELISA

In some circumstances, it may be desirable to assay compositions containing inventive blocking agents in order to ensure that individual blocking agent components (e.g., individual IgE-binding peptides, small molecules, or other agents) are present in the composition. In certain preferred embodiments of the invention, such assays are performed using well known competitive ELISA techniques (see, for example, Alenius et al., J. Immunol. 156:1618, 1996; Chen et al., Clin. Exp. Alleg. 26:406, 1996; and Bayard et al., Immunol. Invest. 28:323, 1999; each of which is incorporated herein by reference). In general, each component is separately attached to a solid support, for example in an individual well of a multi-well plate. Antibodies that recognize each component are then exposed to the solid support(s) in the presence or absence of the composition being studied. It will be appreciated that since, by definition, each component of the inventive binding agent contains a single IgE binding site, such antibodies are obtainable from antigen-sensitive populations (see, for example, Example 1). If the composition contains every component, then competitive inhibition of antibody binding to the solid support should be detected for each and every anti-component antibody. On the other hand, if the composition lacks one or more components, then competitive inhibition of antibody binding will not be observed for those antibodies that recognize the missing components. The assays performed in the absence of the composition under analysis provide a control to ensure that antibody binding can be tested for every component attached to a solid support.

T-Cell Proliferation

Although it is not required that components of an inventive blocking agent be able to stimulate T cell proliferation, it may in some cases be desirable to determine whether the components have such activity. For example, in the case of peptide inducing agents with no intact IgE binding sites but with T-cell epitopes for immunomodulating the immune response away from a Th2 response, a T-cell proliferation assay would be useful in deterring the inducing agent's ability to stimulate T cells and down regulate the Th2 response. T cell proliferation assay techniques are well known in the art (see, for example, Counsell et al., J. Allergy Clin. Immunol. 98:884, 1996; incorporated herein by reference).

### Example 6

### Mimeotope Binding Agents

Many preferred embodiments of the present invention rely on peptides as blocking agents or components thereof. Often, such peptides correspond to a portion of a naturally-occurring polypeptide antigen. While peptides corresponding to a portion of a naturally-occurring antigen can be very effective in blocking IgE binding to linear epitopes on that antigen, it can sometimes be difficult to identify peptides that block IgE binding to conformational epitopes on the antigen. Also, not all antigens of interest are polypeptide antigens. Furthermore, in some cases, it may be desirable to utilize non-peptide binding agents. Accordingly, it may be desirable to identify peptides other than linear sequence peptides and/or non-peptide compounds that block binding of selected IgE molecules to antigens of interest.

In general, compounds that block binding of a given IgE to a particular antigen epitope but are not identical in structure to that epitope are referred to herein as “mimeotopes”. The techniques described herein can be effectively employed to identify mimeotopes for any selected anti-antigen IgE. In particular, a monclonal antibody derived from the anti-antigen IgE is contacted with the antigen in the presence or absence of one or more compounds to be tested for mimeotope activity, for example as described above in Examples 4 and/or 5. Methods of producing monoclonal antibodies of anti-antigen IgE molecules are described in U.S. patent application Ser. No. 09/141,220; 09/240,557; and 09/494,096, filed Aug. 27, 1998, Jan. 29, 1999, and Jan. 28, 2000, respectively; each of which is incorporated herein by reference. If inhibition of IgE binding (and/or basophil histamine release) is observed, then the compound whose presence correlates with the inhibition is designated as an effective mimeotope for purposes of the present invention. Preferably, at least about 20%, 30%, 40%, 50%, 60%, 70%, 80%, 90%, 95%, 97%, 98%, and 99% inhibition of IgE binding and/or basophil histamine release is observed.

Those of ordinary skill in the art will readily appreciate that any compound or collection of compounds can be screened for mimeotope activity as described herein. For example, a library of peptides of all possible sequences could be created or screened. Alternatively or additionally, any of a variety of collections of small molecules and/or peptide mimics (i.e., peptide-like compounds containing one or more non-natural amino acids or other modifications) could be screened.

### Example 7

### Modulation of Peanut Allergy in Mouse Model using a Peptide Mixture

The purpose of this experiment was to assess the therapeutic effect of a peptide mixture representing ten Ara h 2 IgE binding epitopes on Ara h 2-sensitized mice. This Example illustrates passive desensitization using a mixture of peptide derived from Ara h 2 protein. Each of the peptides of the mixture has only one IgE binding site so that it is unable to cross-link IgE molecules on the surface of mast cells.

Materials and Methods

C3H/HeJ mice were sensitized intraperitoneally (IP) with intact Ara h 2 protein (0.5 mg/mouse) and then boosted at week 3. The mice were then challenged ip at week 5 with Ara h 2 (1 mg/mouse). Each of the mice was challenged with either native (N) Ara h 2 protein or reduced (R) Ara h 2 protein (i.e., only linear epitopes). One group of mice received two IP injections of an Ara h 2 peptide mixture (2000 g), and a second group received PBS 24 hours and 6 hours prior to challenge. The Ara h 2 peptide mixture contains ten 20-amino acid peptides selected from Table 4, each of which contains a single IgE binding epitope. Ara h 2-specific IgE levels were measured at week 3, one day before boosting and at week 5, one day before challenge. Anaphylactic symptoms were evaluated utilizing a standardized scoring system (0=no symptoms; 1=scratching and rubbing around the nose and head; 2=puffiness around the eyes and mouth, pilar erecti, reduced activity, and/or decreased activity with increased respiratory rate; 3=wheezing, labored respiration, and cyanosis around the mouth and the tail; 4=no activity after prodding or tremor and convulsion; and 5=death). Rectal temperature and plasma histamine levels were also determined.

Results

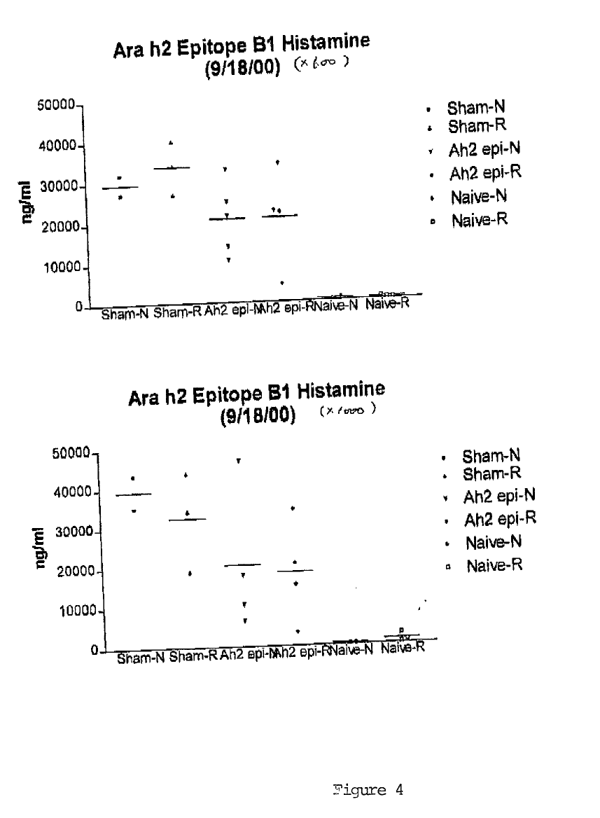

Allergen-specific levels were equivalent in all Ara h 2 sensitized groups at weeks 3 and 5 prior to treatment (Table 6). Following challenge, mice in the PBS sham-treated group exhibited severe anaphylactic reactions, accompanied by reduced rectal temperatures, and elevated plasma histamine levels. Mice in the peptide mixture-treated group showed only mild reactions. Rectal temperatures also dropped, but were higher than those in sham-treated mice. Plasma histamine levels were 30% lower than those observed in the sham-treated group (FIG. 4).

These results suggest that immunotherapy with peptides containing a single IgE epitope prior to allergen challenge may be a potentially beneficial approach to treatment of peanut allergy.

TABLE 6
Challenge of Ara h2 Epitope Experiment 1B

| | | IgE | IgE | | | | | |
|---|---|---|---|---|---|---|---|---|
| Group | No. | ng/ml(1) | ng/ml(2) | Temp. | Score | Histamine(1) | Histamine(2) | Note |
| Sham-N | 1.1 | 430 | 453 | 33.6 | 3 | 27138 | 35212 | |
| | 1.2 | 495 | 531 | 33.6 | 3 | 31932 | 43495 | |
| Average | | 462 | 492 | 33.6 | 3 | 29535 | 39553.5 | |
| Sham-R | 2.1 | 591 | 588 | 34 | 3 | 27130 | 34323 | |
| | 2.2 | 381 | 552 | 34.3 | 3 | 40211 | 44020 | |
| | 2.3 | 441 | 600 | 33.4 | 3 | 34130 | 19135 | |
| Average | | 471 | 546 | 33.9 | 3 | 33823.6667 | 32492.667 | |
| Ah2 epi-N | 3.1 | 420 | 598 | 35.2 | 1 | 14064 | 6540 | |
| | 3.2 | 383 | 456 | 34 | 1 | 21601 | 18002 | |
| | 3.3 | 383 | 636 | 35.1 | 1 | 10637 | 10540 | |
| | 3.4 | 298 | 483 | 34.2 | 1 | 25196 | 409 | |
| | 3.5 | 626 | 907 | 34.2 | 1 | 32995 | 46983 | |
| Average | | 422 | 616 | 34.54 | 1 | 20898.6 | 20516.25 | |
| Ah2 epi-R | 4.1 | 476 | 536 | 33.6 | 4 | | | Dead |
| | 4.2 | 396 | 642 | 37.7 | 3 | 22406 | 15498 | |
| | 4.3 | 247 | 532 | 36.8 | 1 | 4506 | 3413 | |
| | 4.4 | 524 | 678 | 33.9 | 3 | 34329 | 34381 | |
| | 4.5 | 378 | 389 | 33.5 | 3 | 22821 | 20816 | |
| Average | | 404 | 555 | 34.3 | 2.8 | 21015.5 | 18527 | |
| Naive-N | 5.1 | 55 | 58 | 37.1 | 0 | 314 | 412 | |
| | 5.2 | 55 | 42 | 37.8 | 0 | 641 | 293 | |
| Average | | 55 | 50 | 37.45 | 0 | 477.5 | 352.5 | |
| Naive-R | 6.1 | 50 | 44 | 37.8 | 0 | 675 | 2489 | |
| | 6.2 | 45 | 39 | 38.6 | 0 | 296 | 21480 | |
| | 6.3 | 49 | 72 | 38.5 | 0 | 280 | 711 | |
| Average | | 48 | 51 | 38.3 | 0 | 417 | 1600 | |

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

What is claimed is:

1. A method of preventing an allergic response, the method comprising the steps of:
providing an individual allergic to an antigen; and
administering to the individual an agent that blocks the antigen binding sites on offending IgE.

2. A method of preventing an allergic response, the method comprising the steps of:
providing an individual allergic to an antigen; and
administering to the individual an agent that blocks the antigen binding sites on IgE directed against the immunodominant epitopes of the antigen.

3. A method of immunotherapy, the method comprising the steps of:
providing an individual allergic to an antigen;
administering to the individual an agent that blocks the antigen binding sites on offending IgE; and
administering to the individual a composition that comprises the antigen.

4. The method of claim 1 wherein the antigen is an allergen.

5. The method of claim 1 wherein the antigen is a food allergen.

6. The method of claim 5 wherein the food allergen is a peanut allergen.

7. The method of claim 6 wherein the peanut allergen is selected from the groups consisting of Ara h 1, Ara h 2, and Ara h 3.

8. The method of claim 5 wherein the food allergen is a milk allergen.

9. The method of claim 1 wherein the antigen is a shellfish allergen.

10. The method of claim 1 wherein the antigen is an environmental allergen.

11. The method of claim 10 wherein the allergen is a grass pollen.

12. The method of claim 10 wherein the allergen is a tree pollen.

13. The method of claim 1 wherein the antigen is latex.

14. The method of claim 1 wherein the antigen is a drug.

15. The method of claim 1 wherein the antigen is a pollen.

16. The method of claim 1 wherein the antigen is ovalbumin.

17. The method of claim 1 wherein the antigen is an insect venom antigen.

18. The method of claim 1 wherein the antigen is an antigen with predominately linear epitopes.

19. The method of claim 1 wherein the agent is a peptide.

20. The method of claim 1 wherein the agent is a collection of peptides.

21. The method of claim 20 wherein the peptides are designed so that the peptide cannot cross-link IgE.

22. The method of claim 1 wherein the agent is an antibody.

23. The method of claim 1 wherein the agent is a monovalent immunoglobulin.

24. The method of claim 1 wherein the agent blocks the antigen-binding sites of substantially all IgE.

25. The method of claim 1 wherein the agent specifically blocks the antigen-binding sites of substantially all IgE.

26. The method of claim 1 wherein the agent specifically blocks a subset of antigen-binding sites of IgE.

27. The method of claim 1 wherein the agent specifically blocks a subset of antigen-binding sites of IgE that bind the antigen.

28. The method of claim 1 wherein the agent comprises an agent and an adjuvant.

29. The method of claim 1 wherein the agent comprises an agent and a cytokine.

30. The method of claim 3, wherein the composition comprises an antigen and an adjuvant.

31. The method of claim 3, wherein the composition comprises an antigen and a cytokine.

32. The method of claim 3, wherein the composition comprises an antigen and an inducing agent.

33. The method of claim 32, wherein the inducing agent is a peptide.

34. The method of claim 33, wherein the inducing agent is a peptide having at least one T-cell epitope.

35. The method of claim 3, wherein the composition comprises a protein antigen.

36. The method of claim 30 or 31, wherein the adjuvant suppresses a Th2 response.

37. The method of claim 30 or 31, wherein the adjuvant leads to a Th1 response.

38. The method of claim 3, wherein the composition is in a form suitable for standard immunotherapy.

39. The method of claim 3, wherein the composition is in a from suitable for rush immunotherapy.

40. The method of claim 3 wherein the step of administering the composition is performed within one month of the step of administering the agent.

41. The method of claim 3 wherein the step of administering the composition is performed within one week of the step of administering the agent.

42. The method of claim 3 wherein the step of administering the composition is performed within 48 hours of the step of administering the agent.

43. The method of claim 3 wherein the step of administering the composition is performed within 24 hours of the step of administering the agent.

44. The method of claim 3 wherein the step of administering the composition is performed within 8 hours of the step of administering the agent.

45. The method of claim 3 wherein the step of administering the composition is performed within 2 hours of the step of administering the agent.

46. The method of claim 3 wherein the step of administering the composition is performed within 1 hours of the step of administering the agent.

47. The method of claim 4 wherein the step of administering the composition is performed within 30 minutes of the step of administering the agent.

48. A composition comprising an agent that blocks the antigen binding site of an offending IgE.

49. The composition of 48 wherein the agent is a peptide.

50. The composition of 49 wherein the peptide has one or zero IgE binding sites.

51. The composition of 50 wherein the peptide has at least one T-cell epitope.

52. The composition of 48 wherein the agent is collection of peptides.

53. The composition of 48 wherein the agent is a mimeotope.

54. The composition of 48 wherein the agent is a small molecule.

55. A composition comprising an agent that blocks the antigen binding site of an offending IgE and a composition including an antigen that is bound by the offending IgE.

56. A composition comprising an agent that blocks the antigen binding site of an offending IgE in an allergic response.

57. The composition of claim 55 or 56, wherein the agent is a collection of peptides, wherein each peptide no more than one IgE binding site.

58. The composition of claim 55 or 56, wherein the agent is a collection of peptides, wherein each peptide contains only one IgE binding site.

59. The composition of claim 55 or 56, wherein the agent binds but does not cross-link IgE molecules.

60. The composition of claim 55 or 56, wherein the agent is a small molecule.

61. A kit comprising an agent that blocks the antigen binding site of an offending IgE and a composition including an antigen that is bound by the offending IgE suitable for administration to an individual.

62. The kit of claim 61 further comprising a means for administering the agent and the antigen-containing composition.

63. The kit of claim 61 wherein the agent is in the form of an elixir.

64. The kit of claim 61 wherein the agent is in the form of a solid.

65. The kit of claim 61 wherein the agent is in the form of a time-release formulation.

66. The kit of claim 61 wherein the agent is in the form of a depot injectable formulation.

67. The kit of claim 61 wherein the composition including an antigen is in the form of an elixir.
